(19) United States
(12) Patent Application Publication
(10) Pub. No.: US 2018/0363000 A1
(43) Pub. Date: Dec. 20, 2018

(54) AAV-ANTI PCSK9 ANTIBODY CONSTRUCTS AND USES THEREOF

(71) Applicant: The Trustees of the University of Pennsylvania, Philadelphia, PA (US)

(72) Inventors: James M. Wilson, Philadelphia, PA (US); Frank Jacobs, Philadelphia, PA (US); Suryanrayan Somanthan, Downingtown, PA (US)

(21) Appl. No.: 16/061,086

(22) PCT Filed: Dec. 14, 2016
(86) PCT No.: PCT/US16/66631

§ 371 Date: Jun. 11, 2018

Related U.S. Application Data
(60) Provisional application No. 62/267,233, filed on Dec. 14, 2015.

(51) Int. Cl. C12N 15/85; C07K 14/015; C07K 16/40; A61P 9/00; A61P 3/06

(52) U.S. Cl. CPC C12N 15/8509; C07K 14/015; A61K 2039/505; A61P 9/00; A61P 3/06; C07K 16/40

(57) ABSTRACT

Compositions and methods are provided for lowering cholesterol in a subject are provided. An adeno-associated viral vector is provided which includes a nucleic acid molecule comprising a sequence encoding anti-PCSK9 antibody. In desired embodiments, the subject is a human.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “16-7728PCT_Seq_Listing_ST25.txt”.

### BACKGROUND OF THE INVENTION

The World Health Organization estimates that estimated 17.5 million people died from CVDs in 2012, representing 31% of all global deaths. Of these deaths, an estimated 7.4 million were due to coronary heart disease. The most commonly used drugs, statins, target cholesterol production by inhibiting HMG-CoA reductase. However, most high risk subjects on statins with pre-existing coronary artery disease or diabetes fail to achieve their target LDL-c levels. As such an unmet need exists for this high-risk group with hypercholesterolemia.

Increasing hepatic LDLR expression has been demonstrated to be an effective strategy for lowering plasma LDL-c levels in subjects with dyslipidemia and hypercholesterolemia. Human genetic studies demonstrated that subjects with gain-of-function mutations in Proprotein convertase subtilisin/kexin type 9 (PCSK9) had a phenotype similar to familial hypercholesterolemia; in contrast, individuals lacking PCSK9 expression had very low LDL-c but are otherwise normal. Subsequent studies unearthed an unknown PCSK9 function that resulted in its binding to LDLR that resulted in receptor degradation in lysosomes. Additionally, statin treatment via increases in activity/nuclear translocation of SREBP-2 enhances both LDLR and PCSK9 expression. This may contribute to why subjects on statins fail to achieve their target LDL-c levels. These studies have led to the development of PCSK9 inhibition strategies using monoclonal antibodies or RNAi in the next phase of cholesterol lowering drugs. Yet, a caveat of these strategies is the biweekly dosing regimen required to achieve target LDL-c levels. Increasing the interval to 4 weeks significantly reduces the efficacy of PCSK9 monoclonal antibody therapy. At issue is compliance of subjects to undergo regular clinical visits for antibody drug administrations. AAV delivered monoclonal antibody therapy has been demonstrated to achieve long term stable expression and protection against infectious diseases. This approach has the advantage of avoiding the pitfalls associated with repeated delivery of mABs. Studies have demonstrated the validity of this approach to generate high level expression of antibodies in serum of both mice and nonhuman primates (NHPs).

Therefore, compositions useful for expressing anti-PCSK9 antibodies in subjects, are needed.

### SUMMARY OF THE INVENTION

Novel engineered Proprotein convertase subtilisin/kexin type 9 (PCSK9) constructs are provided herein. In one aspect, an adeno-associated viral vector comprising an AAV capsid and at least one expression cassette is provided. The at least one expression cassette comprises nucleic acid sequences encoding an anti-PCSK9 antibody and expression control sequences that direct expression of the anti-PCSK9 antibody sequences in a host cell. In one embodiment, the anti-PCSK9 antibody has the sequence of SEQ ID NO: 1.

In another aspect, the anti-PCSK9 antibody includes variable chain regions (heavy and/or light) from a known monoclonal anti-PCSK9 antibody in combination with constant chain regions (heavy and/or light) from a heterologous antibody backbone.

In another aspect, the anti-PCSK9 antibody includes the complementarity determining regions (CDRs) from a known monoclonal anti-PCSK9 antibody with the remaining antibody sequences derived from a heterologous antibody backbone.

In another aspect, the anti-PSCK9 antibody is a humanized antibody which includes CRDs from a known monoclonal anti-PCSK9 antibody, with the remaining antibody sequences derived from a human.

In another aspect, the anti-PSCK9 antibody is a known monoclonal anti-PCSK9 antibody. In one embodiment, the anti-PCSK9 antibody is a human monoclonal antibody. In another embodiment, the anti-PCSK0 antibody is evolocumab. In yet another embodiment, the anti-PCSK9 antibody is alirocumab.

In yet another aspect, a viral vector is provided which includes at least one expression cassette. The expression cassettes comprise nucleic acid sequences encoding one or more of SEQ ID NO: 2, SEQ ID NO: 3, SEQ ID NO: 4 and SEQ ID NO: 5, and expression control sequences that direct expression of an anti-PSCK9 antibody comprising the specified sequences in a host cell. In one embodiment, the nucleic acid sequences encoding the anti-PCSK9 antibody comprise SEQ ID NO: 6 or a sequence sharing at least 70% identity therewith.

In another aspect, an adeno-associated virus (AAV) vector comprising an AAV8 capsid and at least one expression cassette is provided. The at least one expression cassette includes variable chain regions (heavy and/or light) from a known monoclonal anti-PCSK9 antibody in combination with constant chain regions (heavy and/or light) from a heterologous antibody backbone. In one embodiment, the nucleic acid sequences encoding the anti-PCSK9 antibody of SEQ ID NO: 1, or a sequence sharing at least 95% identity therewith, inverted terminal repeat sequences and expression control sequences that direct expression of the antibody in a host cell.

In another aspect, an adeno-associated virus (AAV) vector comprising an AAV9 capsid and at least one expression cassette is provided. The at least one expression cassette includes variable chain regions (heavy and/or light) from a known monoclonal anti-PCSK9 antibody in combination with constant chain regions (heavy and/or light) from a heterologous antibody backbone. In one embodiment, the nucleic acid sequences encoding the anti-PCSK9 antibody of SEQ ID NO: 1, or a sequence sharing at least 95% identity therewith, inverted terminal repeat sequences and expression control sequences that direct expression of the antibody in a host cell.

In yet another aspect, a pharmaceutical composition is provided which includes a pharmaceutically acceptable carrier, and at least a viral vector as described herein.

In another aspect, a method for lowering cholesterol in a subject is provided. The method includes administering any of the compositions described herein to a subject in need thereof. In one embodiment, the composition is readministered at a later time point.

In another aspect, a method for increasing LDLr in a subject is provided. The method includes administering any of the compositions described herein to a subject in need thereof.

In another aspect, a novel antibody which includes variable chain regions (heavy and/or light) from a known monoclonal anti-PCSK9 antibody in combination with constant chain regions (heavy and/or light) from a heterologous antibody backbone as described herein is provided.

Other aspects and advantages of the invention will be readily apparent from the following detailed description of the invention.

### BRIEF DESCRIPTION OF THE DRAWINGS

FIGS. 1A and 1B demonstrate that AAV8.hPCSK9mAb administration decreases cholesterol levels in mice. C57B/6 mice (N=2) were administered with 1×10¹¹ GC of AAV8.TBG.hPCSK9mAb. (A) Antibody levels in serum before and 14 days after vector administration. (B) Total cholesterol levels before and at days 14, 28 and 42 post vector. Values are expressed as mean±SEM.

FIGS. 2A to 2D demonstrate that AAVhPCSK9mAb administration leads to a dose dependent decrease in cholesterol levels. LAHB (LDLR+/−, APOBEC−/−, hApoB-Tg) mice (N=3/group) were administered with increasing doses of AAVhPCSK9mAb. (A) hPCSK9mAB levels before and at day 7 and 30 following vector administration. (B) Serum cholesterol levels in vector administered animals. (C) Endogenous mouse LDLR expression levels in vector treated animals. (D) Serum non-HDL cholesterol levels in vector administered animals. This graph includes an additional control with mock antibody (solid down pointing triangle). At the completion of study, animals were sacrificed and total liver lysates from two representative animals were run on a 4-12% gradient SDS-PAGE gel and probed with a polyclonal antibody to LDLR. Lysate from mice lacking LDLR expression (DKO) is shown along with those that received increasing doses of vector. Values are expressed as mean±SEM.

FIG. 3 demonstrates that AAV expressed hPCSK9mAb is functional and reduces cholesterol level. hPCSK9mAb was purified from RAG−/− mice previously administered with 1E11 GC of AAVhPCSK9. Purified antibody was concentrated and then administered i.v. to LAHB (LDLR+/−) mice. Serum was collected by retroorbital bleeds and evaluated for plasma lipids.

FIG. 4 is a graph showing total plasma cholesterol (mg/dl) in mice which were injected with 5×10¹⁰ of either AAV8 hLDLr, or AAV8 hLDLr-H306Y, or AAV8 hLDLr-L318D. On day 42 mice received AAV9 PCSK9 at either a high dose of 1×10¹¹ or low of 1×10¹⁰. Total cholesterol was measured at days 0, 7 and 21 after receiving AAV9/PCSK9.

FIG. 5 is a graph showing hPCSK9 expression in mice co-injection of 3×10¹⁰ AAV9/PCSK9 and 5×10¹⁰ of either AAV8/hLDLr, or AAV8/hLDLr-H306Y (gain of function mutant; binds PCSK9 better), or AAV8/hLDLr-L318D (loss of function mutant; hypothetically doesn't or poorly binds PCSK9). A positive control for AAV9/PCSK9 only and AAV8/hLDLr only were also injected. PCSK9 expression was assayed with ELISA.

FIG. 6 is a graph showing plasma cholesterol (mg/dl) for the mice described in FIG. 5. Cholesterol was measured at days 0, 7 and 21.

FIG. 7 is an alignment of the PCSK9 heavy chain and VRC01 heavy chain.

FIG. 8 is an alignment of the PCSK9 light chain and VRC01 light chain.

FIG. 9 shows the amino acid sequence of a PCSK9 antibody construct described herein (SEQ ID NO: 1).

FIG. 10 is a plasmid map of pAAV.CMV.PCSK9.mAB

FIG. 11 is a plasmid map of pAAV.TBG.PCSK9.mAB

FIG. 12 is a graph showing that AAV9.TBG.PCSK9.mAB reduces serum non-HDL cholesterol when administered to DKO mice. Percent reduction in serum non-HDL cholesterol 30 days post vector administration in animals dosed with PCSK9.mAB or the control FI6 antibody. Levels are from averages from 5 animals with SD.

### DETAILED DESCRIPTION OF THE INVENTION

The compositions and regimens described herein are useful for delivery of anti-PCSK9 immunoglobulin constructs to a subject in need thereof. The compositions and regimens are useful for treating lowering serum cholesterol and treating hyperlipidemia and increasing levels of LDLr.

Proprotein convertase subtilisin kexin type 9 (PCSK9) (also known as NARC-1, IgE, dickkopf-related protein 1 (DKK1), Complement 5 (C5), sclerostin (SOST) and GMCSF receptor) was identified as a protein with a genetic mutation in some forms of familial hypercholesterolemia. PCSK9 is synthesized as a zymogen that undergoes autocatalytic processing at a particular motif in the endoplasmic reticulum. Population studies have shown that some PCSK9 mutations are “gain-of-function” and are found in individuals with autosomal dominant hypercholesterolemia, while other “loss-of-function” (LOF) mutations are linked with reduced plasma cholesterol. Morbidity and mortality studies in this group clearly demonstrated that reducing PCSK9 function significantly diminished the risk of cardiovascular disease.

As used herein, the term “subject” means a mammalian animal, including a human, a veterinary or farm animal, a domestic animal or pet, and animals normally used for clinical research. In one embodiment, the subject of these methods and compositions is a human. Still other suitable subjects include, without limitation, murine, rat, canine, feline, porcine, bovine, ovine, non-human primate and others. As used herein, the term “subject” is used interchangeably with “patient”.

The term “immunoglobulin” is used herein to include antibodies, functional fragments thereof, FABs, scFvs, single domain antibodies, DARTs, F(ab′)2, BITEs, and immunoadhesins. These antibody fragments or artificial constructs may include a single chain antibody, a Fab fragment, a univalent antibody, a bivalent of multivalent antibody, or an immunoadhesin. The binding or neutralizing antibody construct may be a monoclonal antibody, a “humanized” antibody, a multivalent antibody, or another suitable construct. An “immunoglobulin molecule” is a protein containing the immunologically-active portions of an immunoglobulin heavy chain and immunoglobulin light chain covalently coupled together and capable of specifically combining with an antigen. Immunoglobulin molecules are of any type (e.g., IgG, IgE, IgM, IgD, IgA and IgY), class (e.g., IgG1, IgG2, IgG3, IgG4, IgA1 and IgA2) or subclass. The terms “antibody” and “immunoglobulin” may be used interchangeably herein. An “immunoglobulin heavy chain” is a polypeptide that contains at least a portion of the antigen binding domain of an immunoglobulin and at least a portion of a variable region of an immunoglobulin heavy chain. Thus, the immunoglobulin derived heavy chain has significant regions of amino acid sequence homology with a member of the immunoglobulin gene superfamily. For example, the heavy chain in a Fab fragment is an immunoglobulin-derived heavy chain. An “immunoglobulin light chain” is a polypeptide that contains at least a portion of the antigen binding domain of an immunoglobulin and at least a portion of the variable region. Thus, the immunoglobulin-derived light chain has significant regions of amino acid homology with a member of the immunoglobulin gene superfamily. An “immunoadhesin” is a chimeric, antibody-like molecule that combines the functional domain of a binding protein, usually a receptor, ligand, cell-adhesion molecule, or 1-2 immunoglobulin variable domains with immunoglobulin constant domains, usually including the hinge or GS linker and Fc regions. A “fragment antigen-binding” (Fab) fragment” is a region on an antibody that binds to antigens. It is composed of one constant and one variable domain of each of the heavy and the light chain. With respect to immunoglobulins or antibodies as described herein, each fragment of an immunoglobulin coding sequence may be derived from one or more sources, or synthesized. Suitable fragments may include the coding region for one or more of, e.g., a heavy chain, a light chain, and/or fragments thereof such as the constant or variable region of a heavy chain (CH1, CH2 and/or CH3) and/or or the constant or variable region of a light chain. Alternatively, variable regions of a heavy chain or light chain may be utilized. Where appropriate, these sequences may be modified from the “native” sequences from which they are derived, as described herein. As used herein, the term “immunoglobulin construct” refers to any of the above immunoglobulins or fragments thereof which are encoded by and included in the expression cassettes and viral vectors described herein.

Antibodies may exist in a variety of forms including, for example, polyclonal antibodies, monoclonal antibodies, camelid heavy chain only (V_{H}H) antibodies, intracellular antibodies (“intrabodies”), recombinant antibodies, multispecific antibody, antibody fragments, such as, Fv, Fab, F(ab)₂, F(ab)₃, Fab′, Fab′-SH, F(ab′)₂, single chain variable fragment antibodies (scFv), tandem/bis-scFv, Fc, pFc′, scFvFc (or scFv-Fc), disulfide Fv (dsfv), bispecific antibodies (bc-scFv) such as BiTE antibodies; humanized camelid antibodies, resurfaced antibodies, humanized antibodies, shark antibodies, fully human antibodies, single-domain antibody (sdAb, also known as NANOBODY®), chimeric antibodies, chimeric antibodies comprising at least one human constant region, and the like. “Antibody fragment” refers to at least a portion of the variable region of the immunoglobulin that binds to its target, e.g., the PCSK9 protein.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous. With regard to the antibodies described herein, in one embodiment the constant regions of the heavy and/or light chain are from a different source than the variable regions of the heavy and/or light chain. Thus, with reference to each other, said constant and variable regions are heterologous. The different sources may be from the same species or different species.

As used herein, an “expression cassette” refers to a nucleic acid molecule which comprises an immunoglobulin coding sequence (e.g., an immunoglobulin variable region, an immunoglobulin constant region, a full-length light chain, a full-length heavy chain or another fragment of an immunoglobulin construct, or combinations thereof), promoter, and may include other regulatory sequences therefor, which cassette may be delivered via a genetic element (e.g., a plasmid) to a packaging host cell and packaged into the capsid of a viral vector (e.g., a viral particle). Typically, such an expression cassette for generating a viral vector contains the immunoglobulin sequences described herein flanked by packaging signals of the viral genome and other expression control sequences such as those described herein. In one embodiment, the expression cassettes contain at least one of an AAV5′ ITR and an AAV 3′ ITR. In some embodiments, the terms “rAAV genome”, “viral genome” and “vector genome” are used interchangeably. In one embodiment, the rAAV genome includes a 5′ ITR, a CMV enhancer and promoter, an intron, such as the promega chimeric intron, PCSK9mAB coding sequence, a poly(A) sequence, such as the SV40 poly(A), and a 3′ ITR. In one embodiment, the rAAV genome comprises nt 14 to 3988 of SEQ ID NO: 9. In another embodiment, the rAAV genome includes a 5′ ITR, an alpha mic/bik enhancer, a TBG promoter, an intron, such as the promega chimeric intron, PCSK9mAB coding sequence, a poly(A) sequence, such as the rabbit globin poly(A), and a 3′ ITR. In one embodiment, the rAAV genome comprises nt 20 to 3657 of SEQ ID NO: 10.

In one aspect, an adeno-associated viral (AAV) vector is provided. The AAV vector includes an AAV capsid and at least one expression cassette. The at least one expression cassette includes nucleic acid sequences encoding a novel anti-PCSK9 antibody and expression control sequences that direct expression of the anti-PCSK9 antibody sequences in a host cell. In one embodiment, the antibody comprises an anti-PCSK9 heavy or light chain variable sequence. Various anti-PCSK9 monoclonal antibodies are known or are in development. Certain of the amino acid sequences for the anti-PCSK9 immunoglobulin construct are selected from those which have been published, those which are now, or may become, commercially available, and the coding sequences described herein. Specifically, in a preferred embodiment, the heavy and light chain variable regions are anti-PCSK9 antibody sequences. In one embodiment, the antibody is a fully human antibody. Various anti-PCSK9 antibodies can be the source of the variable chain sequences. An example utilizing such sequences is shown in SEQ ID NO: 1 (also shown in FIG. 9). In one embodiment, the heavy chain variable region is shown in SEQ ID NO: 4. In another embodiment, the light chain variable region is shown in SEQ ID NO: 5.

An example of a desirable source of anti-PCSK9 antibody sequences is evolocumab. Evolocumab is a fully human monoclonal antibody that typically achieves approximately a 60% reduction in LDL cholesterol levels when administered at the doses that were studied in phase 3 trials. Evolocumab is a human IgG2 monoclonal antibody that is designed to bind to PCSK9 and inhibit PCSK9 from binding to LDL receptors on the liver surface, resulting in more LDL receptors on the surface of the liver to remove LDL-C from the blood. See, e.g., http://www.drugbank.ca/drugs/DB09303. The amino acid sequences of the evolocumab heavy and light chains can be found, e.g., http://www.genome.jp/dbget-bin/www_bget?dr:D10557. In one embodiment, the heavy chain variable sequence is that set forth in SEQ ID NO: 4. In one embodiment, the light chain variable sequence is that set forth in SEQ ID NO: 5. In another embodiment, the PCSK9 sequences are complementarity determining regions (CDRs). The CDRs of evolocumab are published with U.S. Pat. No. 8,871,913, which is incorporated herein by reference in its entirety. In one embodiment, the heavy chain sequence is that set forth in SEQ ID NO: 13. In another embodiment, the light chain sequence is that set forth in SEQ ID NO: 14.

Another example of a desirable source of anti-PCSK9 antibody sequences is bococizumab. Bococizumab (RN316/PF-04950615) is a humanized IgG2Δa monoclonal antibody (mAb) that recognizes and binds to the LDLR-binding domain of PCSK9, thus preventing PCSK9-mediated degradation of LDLR, leading to improved LDL clearance and reduction of serum LDL-C levels. In phase 1 and 2a clinical trials in hypercholesterolemic subjects, bococizumab reduced LDL-C levels by up to ˜75% and was generally well tolerated with few subjects discontinuing treatment because of adverse events. See, e.g., Ballantyne et al, The American Journal of Cardiology, 115(9):1212-21 (May 2015). The amino acid sequences of the bococizumab heavy and light chains can be found, e.g., http://www.genome.jp/dbget-bin/www._bget?dr:D1062. In another embodiment, the PCSK9 sequences are complementarity determining regions (CDRs) of bococizumab. The CDRs of bococizumab are published with U.S. Pat. No. 8,080,243, which is incorporated herein by reference in its entirety.

Yet another example of a desirable source of anti-PCSK9 antibody sequences is alirocumab. Alirocumab is a human monoclonal antibody (mAb) of the IgG isotype, having a molecular weight of 146 kDa. See, e.g., Gouni-Berthold and Berthold, Nutrients, 2014 December, 6(12):5517-33. The amino acid sequences of the alirocumab heavy (SEQ ID NO: 11) and light chains (SEQ ID NO: 12) can be found, e.g., http://www.genome.jp/dbget-bin/www_bget?dr:D10335. In another embodiment, the PCSK9 sequences are complementarity determining regions (CDRs) of alirocumab. The CDRs of alirocumab are published with U.S. Pat. No. 8,062,640, which is incorporated herein by reference in its entirety.

Other sources of anti-PCSK9 antibody sequences include RG-7652 (Roche), (see Tingley et al, Heart 2013; 99:A153 doi:10.1136/heartjnl-2013-304613.423 and Gelzleichter et al, Toxicol. Sci. (2014) doi: 10.1093/toxsci/kfu093, which are incorporated herein by reference). Another source of anti-PCSK9 antibody sequences is LY3015014 (Eli Lily) (see, Schroeder et al, J. J Lipid Res. 2015 November; 56(11):2124-32. doi: 10.1194/jlr.M061903. Epub 2015 Sep. 20 which is incorporated herein by reference. See, Sheridan, Phase 3 data for PCSK9 inhibitor wows, Nature Biotechnology 31, 1057-1058 (2013), which is incorporated herein by reference.

Yet another example of a desirable source of anti-PCSK9 antibody sequences is LGT-209 (Novartis).

Another example of a desirable source of anit-PCSK9 antibody sequences is the antibodies described in U.S. Pat. No. 9,029,515, which is incorporated herein by reference in its entirety. In another embodiment, the PCSK9 sequences are complementarity determining regions (CDRs) of the antibodies described in U.S. Pat. No. 9,029,515.

Yet another example of a desirable source of anti-PCSK9 antibody sequences is the antibodies described in International Patent Publication No. WO 2014/107739, which is incorporated herein by reference in its entirety. In another embodiment, the PCSK9 sequences are complementarity determining regions (CDRs) of the antibodies described in WO 2014/107739.

Yet another example of a desirable source of anti-PCSK9 antibody sequences is the antibodies described in US Patent Publication No. US 2012/0195910, which is incorporated herein by reference in its entirety. In another embodiment, the PCSK9 sequences are complementarity determining regions (CDRs) of the antibodies described in US 2012/0195910.

Other anti-PCSK9 antibodies useful herein are known in the art. See, e.g., Catapano et al, The safety of therapeutic monoclonal antibodies: Implications for cardiovascular disease and targeting the PCSK9 pathway, Volume 228, Issue 1, May 2013, Pages 18-28, which is incorporated herein by reference. Such antibodies include 1B20 (Merck & Co.), PF-04950615/RN-316 (Pfizer) and LGT 209 (Novartis), LY3015014 and MPSK3169A (Roche-Genentech, Phase 2). In one embodiment, the anti-PCSK9 antibody is SAR236553/REGN727. In another embodiment, the anti-PCSK9 antibody is mAb1/AMG145. In another embodiment, the anti-PCSK9 antibody is 1B20. In another embodiment, the anti-PCSK9 antibody is PF-04950615/RN-316. In another embodiment, the anti-PCSK9 antibody is LGT209. In another embodiment, the anti-PCSK9 antibody is MPSK3169A. In another embodiment, the anti-PCSK9 antibody is LY3015014.

In one embodiment, the heavy chain constant regions (CH1, CH2 and CH3) are those shown in SEQ ID NO: 2. In another embodiment, various allotypes of human IgG1 or other antibody isotypes can also be used. In another embodiment, the light chain constant region is that shown in SEQ ID NO: 3 or selected from known kappa and lambda chain sequences.

Other sources of heavy and light chain constant regions are also within the scope of the invention. Suitable heavy and light chain constant regions are disclosed in WO 2015/012924, which is incorporated herein by reference in its entirety. Other suitable heavy and light chain constant sequences are disclosed in WO 2015/164723 and WO 2015/175639, each of which is incorporated herein by reference in its entirety.

It is also contemplated that one or more of the immunoglobulin sequences useful herein encompasses variants of the immunoglobulin sequences described herein where modifications and/or substitutions have been made. Such variants include immunoglobulins which share at least 70% identity with any of the sequences described herein, over the nucleic acid or amino acid level. In another embodiment, the immunoglobulin sequences comprising the novel antibody include sequence which share at least 75%, at least 80%, at least 85%, at least 90%, at least 95%, at least 97% or at least 99% identity with any of the sequences described herein, over the nucleic acid or amino acid level. In one embodiment, the anti-PCSK9 coding sequence is SEQ ID NO: 8, or a sequence which shares at least 75%, at least 80%, at least 85%, at least 90%, at least 95%, at least 97% or at least 99% identity therewith.

Such modifications and/or substitutions can be made at the nucleic acid or amino acid level. In one embodiment, the coding sequence of one or more immunoglobulin chain or region is codon optimized.

Once the target and immunoglobulin are selected, the coding sequences for the selected immunoglobulin (e.g., heavy and/or light chain(s)) may be obtained and/or synthesized. Methods for sequencing a protein, peptide, or polypeptide (e.g., as an immunoglobulin) are known to those of skill in the art. Once the sequence of a protein is known, there are web-based and commercially available computer programs, as well as service based companies which back translate the amino acids sequences to nucleic acid coding sequences. See, e.g., backtranseq by EMBOSS, http://www.ebi.ac.uk/Tools/st/; Gene Infinity (http://www.geneinfinity.org/sms/sms_backtranslation.html); ExPasy (http://www.expasv.org/tools/). In one embodiment, the RNA and/or cDNA coding sequences are designed for optimal expression in human cells.

Codon-optimized coding regions can be designed by various different methods. This optimization may be performed using methods which are available on-line (e.g., GeneArt), published methods, or a company which provides codon optimizing services, e.g., DNA2.0 (Menlo Park, Calif.). One codon optimizing method is described, e.g., in US International Patent Publication No. WO 2015/012924, which is incorporated by reference herein in its entirety. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered (e.g., heavy constant, light constant, heavy variable, light variable chains). By using one of these methods, one can apply the frequencies to any given polypeptide sequence, and produce a nucleic acid fragment of a codon-optimized coding region which encodes the polypeptide.

A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In one embodiment, such variants include sequences in which amino acid substitutions have been made to the known anti-PCSK9 variable chain sequences or heterologous backbone sequences described herein. Substitutions may also be written as (amino acid identified by single letter code)-position #-(amino acid identified by single letter code) whereby the first amino acid is the substituted amino acid and the second amino acid is the substituting amino acid at the specified position. The terms “substitution” and “substitution of an amino acid” and “amino acid substitution” as used herein refer to a replacement of an amino acid in an amino acid sequence with another one, wherein the latter is different from the replaced amino acid. Methods for replacing an amino acid are well known to the person skilled in the art and include, but are not limited to, mutations of the nucleotide sequence encoding the amino acid sequence. Methods of making amino acid substitutions in IgG are described, e.g., for WO 2013/046704, which is incorporated by reference for its discussion of amino acid modification techniques.

The term “amino acid substitution” and its synonyms described above are intended to encompass modification of an amino acid sequence by replacement of an amino acid with another, substituting amino acid. The substitution may be a conservative or non-conservative substitution. The term conservative, in referring to two amino acids, is intended to mean that the amino acids share a common property recognized by one of skill in the art. The term non-conservative, in referring to two amino acids, is intended to mean that the amino acids which have differences in at least one property recognized by one of skill in the art. For example, such properties may include amino acids having hydrophobic nonacidic side chains, amino acids having hydrophobic side chains (which may be further differentiated as acidic or nonacidic), amino acids having aliphatic hydrophobic side chains, amino acids having aromatic hydrophobic side chains, amino acids with polar neutral side chains, amino acids with electrically charged side chains, amino acids with electrically charged acidic side chains, and amino acids with electrically charged basic side chains. Both naturally occurring and non-naturally occurring amino acids are known in the art and may be used as substituting amino acids in embodiments. Thus, a conservative amino acid substitution may involve changing a first amino acid having a hydrophobic side chain with a different amino acid having a hydrophobic side chain; whereas a non-conservative amino acid substitution may involve changing a first amino acid with an acidic hydrophobic side chain with a different amino acid having a different side chain, e.g., a basic hydrophobic side chain or a hydrophilic side chain. Still other conservative or non-conservative changes change be determined by one of skill in the art.

In still other embodiments, the substitution at a given position will be to an amino acid, or one of a group of amino acids, that will be apparent to one of skill in the art in order to accomplish an objective identified herein.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, 91%, 92%, 93%, 94%, 95%, 96%, 97%, 98%, 99%, or higher identity over a specified region (e.g., any one of the modified ORFs provided herein when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). As another example, polynucleotide sequences can be compared using Fasta, a program in GCG Version 6.1. Fasta provides alignments and percent sequence identity of the regions of the best overlap between the query and search sequences. For instance, percent sequence identity between nucleic acid sequences can be determined using Fasta with its default parameters (a word size of 6 and the NOPAM factor for the scoring matrix) as provided in GCG Version 6.1, herein incorporated by reference. Generally, these programs are used at default settings, although one skilled in the art can alter these settings as needed. Alternatively, one of skill in the art can utilize another algorithm or computer program that provides at least the level of identity or alignment as that provided by the referenced algorithms and programs. This definition also refers to, or can be applied to, the compliment of a sequence. The definition also includes sequences that have deletions and/or additions, as well as those that have substitutions. As described below, the preferred algorithms can account for gaps and the like. Preferably, identity exists over a region that is at least about 25, 50, 75, 100, 150, 200 amino acids or nucleotides in length, and oftentimes over a region that is 225, 250, 300, 350, 400, 450, 500 amino acids or nucleotides in length or over the full-length of an amino acid or nucleic acid sequences.

Typically, when an alignment is prepared based upon an amino acid sequence, the alignment contains insertions and deletions which are so identified with respect to a reference AAV sequence and the numbering of the amino acid residues is based upon a reference scale provided for the alignment. However, any given AAV sequence may have fewer amino acid residues than the reference scale. In the present invention, when discussing the parental sequence, the term “the same position” or the “corresponding position” refers to the amino acid located at the same residue number in each of the sequences, with respect to the reference scale for the aligned sequences. However, when taken out of the alignment, each of the proteins may have these amino acids located at different residue numbers. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Further, in one embodiment, the nucleic acid molecule includes a heterologous leader sequence for either the heavy chain, the light chain, or both of the anti-PCSK9 antibody sequence(s). In one embodiment, the heterologous leader encodes the sequence MDWTWRILFLVAAATGAHS (SEQ ID NO: 6) and is fused upstream of the heavy chain polypeptides composed of the variable and constant regions. In another embodiment, the heterologous leader encodes the sequence METPAQLLFLLLLWLPDTTG (SEQ ID NO: 7) and is fused upstream of the light chain polypeptides composed of the variable and constant regions. However, another heterologous leader sequence may be substituted for the noted leader sequences. Signal/leader peptides may be the same or different for each the heavy chain and light chain immunoglobulin constructs. These may be signal sequences which are natively found in an immunoglobulin (e.g., IgG), or may be from a heterologous source. Such heterologous sources may be a cytokine (e.g., IL-2, IL12, IL18, or the like), insulin, albumin, β-glucuronidase, alkaline protease or the fibronectin secretory signal peptides, amongst others. Other suitable leader sequences can be found at http://www2.mrc-lmb.cam.ac.uk/vbase/alignments 2.php, which is incorporated herein by reference or designed by the person of skill in the art.

The expression cassette described herein may contain at least one internal ribosome binding site, i.e., an IRES, located between the coding regions of the heavy and light chains. Alternatively the heavy and light chain may be separated by a furin-2a self-cleaving peptide linker (see, e.g., Radcliffe and Mitrophanous, Gene Therapy (2004), 11, 1673-1674, which is incorporated herein by reference).

In one embodiment, the immunoglobulin genes described herein are engineered into a genetic element (e.g., a plasmid) useful for generating AAV vectors which transfer the immunoglobulin construct sequences carried thereon. The selected vector may be delivered to a an AAV packaging cell by any suitable method, including transfection, electroporation, liposome delivery, membrane fusion techniques, high velocity DNA-coated pellets, viral infection and protoplast fusion. Stable packaging cells can also be made. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, N.Y. (2012).

An AAV vector, or expression cassette, as described herein can comprise one or more nucleic acid sequences, each of which encodes one or more of the heavy and/or light chain polypeptides, or other polypeptides, of an anti-PCSK9 immunoglobulin. Suitably, a composition contains one or more AAV vectors which contain all of the polypeptides which form an anti-PCSK9 antibody in vivo. For example, a full-length antibody consists of four polypeptides: two identical copies of a heavy (H) chain polypeptide and two copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. In this respect, an AAV vector as described herein can comprise a single nucleic acid sequence that encodes the two heavy chain polypeptides (e.g., constant and variable) and the two light chain polypeptides of an immunoglobulin construct. Alternatively, the AAV vector can comprise a first expression cassette that encodes the heavy chain constant polypeptides and the heavy chain variable polypeptide, and a second expression cassette that encodes both light chain polypeptides of an immunoglobulin construct. In yet another embodiment, the AAV vector can comprise a first expression cassette encoding a first heavy chain polypeptide, a second expression cassette encoding a second heavy chain polypeptide, a third expression cassette encoding a first light chain polypeptide, and a fourth expression cassette encoding a second light chain polypeptide. In a preferred embodiment, the immunoglobulin sequences are provided in a single expression cassette that includes an IRES or F2A site.

Typically, an expression cassette for an AAV vector comprises an AAV 5′ inverted terminal repeat (ITR), the immunoglobulin coding sequences and any regulatory sequences, and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and 3′ ITRs are used.

Where a pseudotyped AAV is to be produced, the ITRs in the expression cassette are selected from a source which differs from the AAV source of the capsid. In one embodiment, the ITR sequences from AAV2, or the deleted version thereof (ΔITR), are used for convenience and to accelerate regulatory approval. However, ITRs from other AAV sources may be selected.

Each rAAV genome can be then introduced into a production plasmid. In one embodiment, the production plasmid is that described herein, or as described in WO2012/158757, which is incorporated herein by reference. Various plasmids are known in the art for use in producing rAAV vectors, and are useful herein. The production plasmids are cultured in the host cells which express the AAV cap and/or rep proteins. In the host cells, each rAAV genome is rescued and packaged into the capsid protein or envelope protein to form an infectious viral particle.

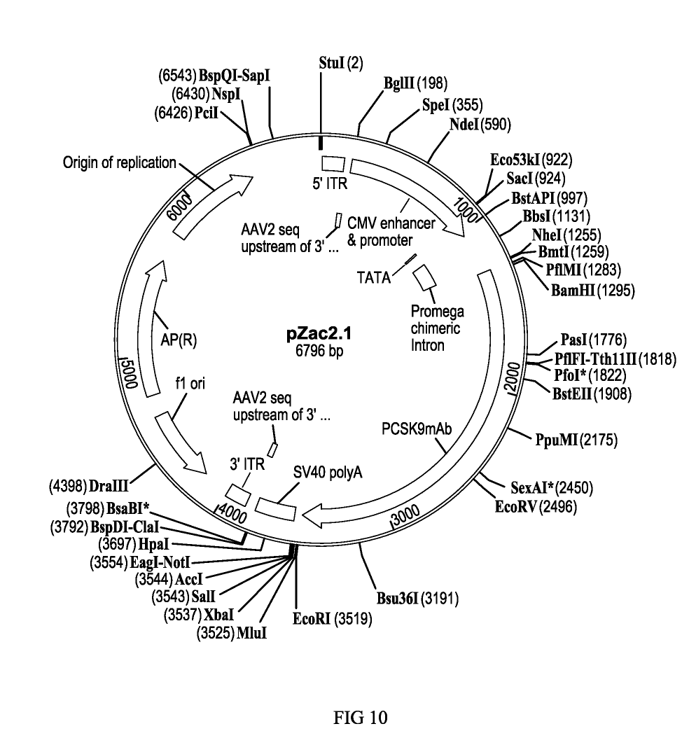

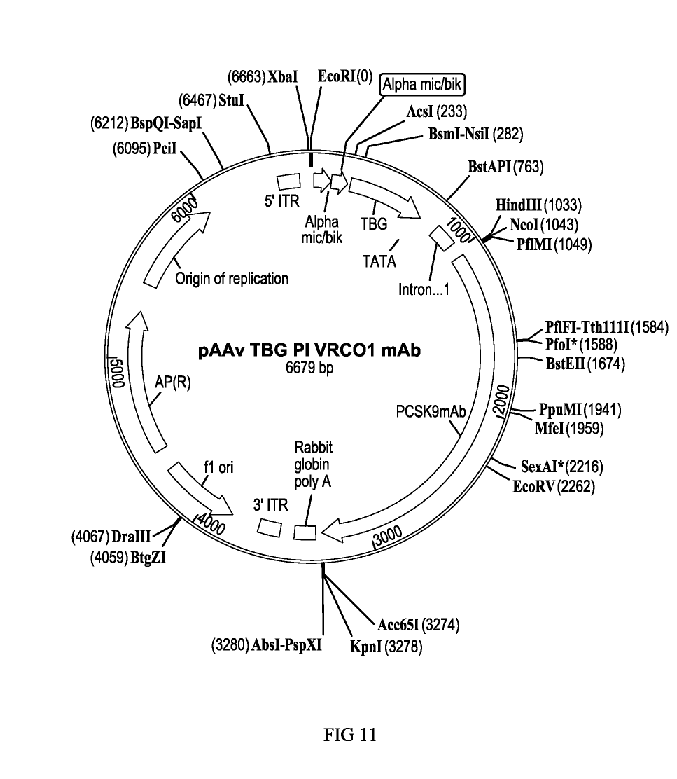

One type of production plasmid is that shown in SEQ ID NO: 9 and FIG. 10, which is termed p2175_pAAV_CMV_PI_PCSK9. This plasmid is used in the examples for generation of the rAAV-PCSK9 vector. Such a plasmid is one that contains a 5′ AAV ITR sequence; a selected promoter; enhancer, sequences encoding the anti-PCSK9 antibody, a polyA sequence; and a 3′ ITR. Such plasmid may also contain a poly A sequence or an intron. Another such production plasmid is shown in FIG. 11 and SEQ ID NO: 10.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety. In one embodiment, the rAAV is an scAAV.

The expression cassette typically contains a promoter sequence as part of the expression control sequences, e.g., located between the selected 5′ ITR sequence and the immunoglobulin construct coding sequence. In one embodiment, expression in liver is desirable. Thus, in one embodiment, a liver-specific promoter is used. Tissue specific promoters, constitutive promoters, regulatable promoters [see, e.g., WO 2011/126808 and WO 2013/04943], or a promoter responsive to physiologic cues may be used may be utilized in the vectors described herein. In another embodiment, expression in muscle is desirable. Thus, in one embodiment, a muscle-specific promoter is used. In one embodiment, the promoter is an MCK based promoter, such as the dMCK (509-bp) or tMCK (720-bp) promoters (see, e.g., Wang et al, Gene Ther. 2008 November; 15(22):1489-99. doi: 10.1038/gt.2008.104. Epub 2008 Jun. 19, which is incorporated herein by reference). Another useful promoter is the SPc5-12 promoter (see Rasowo et al, European Scientific Journal June 2014 edition vol. 10, No. 18, which is incorporated herein by reference). In one embodiment, the promoter is a CMV promoter. In another embodiment, the promoter is a TBG promoter. TBG is a hybrid promoter based on the human thyroid hormone-binding globulin (TBG) promoter and microglobin/bikunin enhancer; it is about 0.8 kb in length (also called liver specific promoter or LSP). This hybrid promoter has been used for liver-specific transgene expression. In certain of the plasmids and vectors described herein, a CB7 promoter is used. CB7 is a chicken β-actin promoter with cytomegalovirus enhancer elements. Alternatively, other liver-specific promoters may be used [see, e.g., The Liver Specific Gene Promoter Database, Cold Spring Harbor, http://rulai.schl.edu/LSPD, alpha 1 anti-trypsin (A1AT); human albumin (Miyatake et al., J. Virol., 71:5124 32 (1997)), humAlb; transthyretin promoter, and hepatitis B virus core promoter, (Sandig et al., Gene Ther., 3:1002 9 (1996))]. Other promoters include the TTR minimal enhancer/promoter and alpha-antitrypsin promoter.

The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter.

The expression cassette may contain at least one enhancer, i.e., CMV enhancer. Still other enhancer elements may include, e.g., an apolipoprotein enhancer, prothrombin enhancer, synthetic enhancer, a zebrafish enhancer, a GFAP enhancer element, microglobin/bikunin enhancer, and liver specific enhancers, woodchuck post hepatitis post-transcriptional regulatory element. Additionally, or alternatively, other , e.g., the hybrid human cytomegalovirus (HCMV)-immediate early (IE)-PDGR promoter or other promoter—enhancer elements may be selected. To enhance expression other elements can be included, such as introns (like promega intron or chimeric chicken globin-human immunoglobulin intron). Other such sequences are known in the art.

In addition to a promoter, an expression cassette and/or a vector may contain other appropriate transcription initiation, termination, enhancer sequences, introns, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product.

These control sequences are “operably linked” to the immunoglobulin construct gene sequences. As used herein, the term “operably linked” refers to both expression control sequences that are contiguous with the gene of interest and expression control sequences that act in trans or at a distance to control the gene of interest.

In still a further aspect, a recombinant adeno-associated virus (AAV) vector is provided for delivery of the anti-PCSK9 antibody constructs and optimized sequences described herein. An adeno-associated virus (AAV) viral vector is an AAV DNase-resistant particle having an AAV protein capsid into which is packaged nucleic acid sequences for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. AAVs may be selected as sources for capsids of AAV viral vectors as identified above. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. No. 7,790,449 and U.S. Pat. No. 7,282,199 (AAV8), WO 2005/033321 and U.S. Pat. No. 7,906,111 (AAV9), and WO 2006/110689, and WO 2003/042397 (rh.10). These documents also describe other AAV which may be selected for generating AAV and are incorporated by reference. In some embodiments, an AAV cap for use in the viral vector can be generated by mutagenesis (i.e., by insertions, deletions, or substitutions) of one of the aforementioned AAV capsids or its encoding nucleic acid. In some embodiments, the AAV capsid is chimeric, comprising domains from two or three or four or more of the aforementioned AAV capsid proteins. In some embodiments, the AAV capsid is a mosaic of Vp1, Vp2, and Vp3 monomers from two or three different AAVs or recombinant AAVs. In some embodiments, an rAAV composition comprises more than one of the aforementioned Caps.

In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3). In one embodiment, the AAV capsid shares at least 95% identity with the AAV8 vp3. In one embodiment, the AAV capsid shares at least 95% identity with the AAV9 vp3. In another embodiment, a self-complementary AAV is used. In one embodiment, it is desirable to utilize an AAV capsid, which shows tropism for the desired target cell, e.g., muscle or liver.

In some of the examples below, an AAV8 vector is described for expressing anti-PCSK9 antibodies in subjects in need of reduction of cholesterol. AAV8 vectors are described, e.g., in International Patent Publication No. WO/2003/052051, which is incorporated herein by reference. In another embodiment, AAV9 vectors are used for expressing anti-PCSK9 antibodies in subject in need of reduction of cholesterol. However, other sources of AAV capsids and other viral elements may be selected, as may other immunoglobulin constructs and other vector elements. Methods of generating AAV vectors have been described extensively in the literature and patent documents, including, e.g., WO 2003/042397; WO 2005/033321, WO 2006/110689; U.S. Pat. No. 7,588,772 B2.

In one embodiment, the source of AAV capsid is selected from an AAV which targets muscle. In another embodiment, the source of AAV capsid is selected from an AAV which targets liver. Suitable AAV may include, e.g, AAV9 [U.S. Pat. No. 7,906,111; US 2011-0236353-A1], rh10 [WO 2003/042397], hu37 [see, e.g., U.S. Pat. No. 7,906,111; US 2011-0236353-A1] and/or AAVrh.64R1. However, other AAV, including, e.g., AAV1, AAV2, AAV3, AAV3B, AAV4, AAV5, AAV6, AAV7 (WO 2003/042937), AAV8 [U.S. Pat. No. 7,790,449; U.S. Pat. No. 7,282,199], and others may be selected for preparing the AAV vectors described herein. See, e.g., Wang et al, The potential of adeno-associated viral vectors for gene delivery to muscle tissue, Expert Opin Drug Deliv. 2014 March; 11(3): 345-364, which is incorporated herein by reference. Other suitable AAV capsids are described in WO 2006/110689 and WO 2003/042937, both of which are incorporated herein by reference.

For packaging an expression cassette or rAAV genome or production plasmid into virions, the ITRs are the only AAV components required in cis in the same construct as the transgene. In one embodiment, the coding sequences for the replication (rep) and/or capsid (cap) are removed from the AAV genome and supplied in trans or by a packaging cell line in order to generate the AAV vector. For example, as described above, a pseudotyped AAV may contain ITRs from a source which differs from the source of the AAV capsid. Additionally or alternatively, a chimeric AAV capsid may be utilized. Still other AAV components may be selected. Sources of such AAV sequences are described herein and may also be isolated or engineered obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank®, PubMed®, or the like.

In one embodiment, a self-complementary AAV is provided. This viral vector may contain a Δ5′ ITR and an AAV 3′ ITR. In another embodiment, a single-stranded AAV viral vector is provided. Methods for generating and isolating AAV viral vectors suitable for delivery to a subject are known in the art. See, e.g., U.S. Pat. No. 7,790,449; U.S. Pat. No. 7,282,199; WO 2003/042397; WO 2005/033321, WO 2006/110689; and U.S. Pat. No. 7,588,772 B2]. In one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The available space for packaging may be conserved by combining more than one transcription unit into a single expression cassette, thus reducing the amount of required regulatory sequences. For example, a single promoter may direct expression of a single cDNA or RNA that encodes two or three or more genes, and translation of the downstream genes are driven by IRES sequences. In another example, a single promoter may direct expression of a cDNA or RNA that contains, in a single open reading frame (ORF), two or three or more genes separated from one another by sequences encoding a self-cleavage peptide (e.g., 2A) and/or a protease recognition site (e.g., furin). The ORF thus encodes a single polyprotein, which, either during or after translation, is cleaved into the individual proteins (such as, e.g., heavy chain and light chain). It should be noted, however, that although these IRES and polyprotein systems can be used to save AAV packaging space, they can only be used for expression of components that can be driven by the same promoter. In another alternative, the transgene capacity of AAV can be increased by providing AAV ITRs of two genomes that can anneal to form head to tail concatamers.

Suitably, the composition of the invention are designed so that AAV vectors carry the nucleic acid expression cassettes encoding the immunoglobulin constructs and regulatory sequences which direct expression of the immunoglobulin thereof in the selected cell. In one embodiment, the selected cell is a liver cell. In one embodiment, the selected cell is a muscle cell.

In one embodiment, following administration of the vectors to the subject, the vectors deliver the expression cassettes to the muscle and express the proteinaceous immunoglobulin constructs in vivo. In another embodiment, the expression cassettes are delivered to the liver. The use of compositions described herein in reducing plasma cholesterol levels are described, as are uses of these compositions for treatment of hyperlipidemia, which may optionally involve delivery of one or more cholesterol-reducing, or LDLr-increasing, agents.

As used herein, when referring to plasma cholesterol, in one embodiment, it is meant total plasma cholesterol levels. In another embodiment, plasma cholesterol refers to non-HDL cholesterol, which is the difference between the total cholesterol concentration and the HDL cholesterol concentration, providing an estimate of cholesterol in the atherogenic particles including IDL, VLDL, Lp(a), and LDL.

Indications for use of the compositions and methods described herein include treatment of adults with heterozygous familial hypercholesterolemia or clinical atherosclerotic cardiovascular disease, who require additional lowering of LDL-cholesterol (LDL-C).

As stated above, a composition may contain a single type of AAV vector as described herein which contains the expression cassette for delivering the novel anti-PCSK9 antibody in vivo. Alternatively, a composition may contain two or more different AAV vectors, each of which has packaged therein different expression cassettes. For example, the two or more different AAV may have different expression cassettes which express immunoglobulin polypeptides which assemble in vivo to form a single functional immunoglobulin. In another example, the two or more AAV may have different expression cassettes which express polypeptides for different targets, e.g., one provides for a functional anti-PCSK9 antibody and a second LDLr-expressing construct.

Also included herein are methods of reducing cholesterol in a subject in need thereof. A regimen as described herein may comprise, in addition to one or more of the compositions described herein, combination with one or more compositions which lowers cholesterol. In one embodiment the composition is administered with an AAV vector which comprises an expression cassette which encodes LDLr. In another embodiment, a composition as described herein is administered with LDLr. In another embodiment, a composition as described herein is administered with a statin.

Other such compositions include anti-PCSK9 antibodies, such as alirocumab. See, e.g., Praluent product literature for usage recommendations, which is incorporated herein by reference.

Suitably, the compositions described herein comprise in an amount effective to reduce free PCSK9 and/or reduce serum cholesterol, one or more AAV suspended in a pharmaceutically suitable carrier designed for delivery to the subject via injection, osmotic pump, intrathecal catheter, or for delivery by another device or route. The pharmaceutical compositions described herein are designed for delivery to subjects in need thereof by any suitable route or a combination of different routes. Direct delivery to the liver (optionally via intravenous, via the hepatic artery, or by transplant), oral, inhalation, intranasal, intrathecal, intratracheal, intraarterial, intraocular, intravenous, intramuscular, subcutaneous, intradermal, and other parental routes of administration. In one example, the composition is formulated for intramuscular delivery.

In yet other aspects, these nucleic acid sequences, vectors, expression cassettes and rAAV viral vectors are useful in a pharmaceutical composition, which also comprises a pharmaceutically acceptable carrier, buffer, diluent and/or adjuvant, etc. Such pharmaceutical compositions are used to express the anti-PCSK9 antibodies through delivery by such recombinantly engineered AAVs or artificial AAVs. In another aspect, a pharmaceutical composition is provided which includes a novel antibody of the invention and a pharmaceutically acceptable carrier, buffer, diluent and/or adjuvant, etc.

To prepare these pharmaceutical compositions containing the nucleic acid sequences, vectors, expression cassettes and rAAV viral vectors or antibodies, the sequences or vectors or viral vector is preferably assessed for contamination by conventional methods and then formulated into a pharmaceutical composition suitable for administration to the subject. Such formulation involves the use of a pharmaceutically and/or physiologically acceptable vehicle or carrier, particularly one suitable for administration to the muscle, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20.

In one exemplary specific embodiment, the composition of the carrier or excipient contains 180 mM NaCl, 10 mM NaPi, pH7.3 with 0.0001%-0.01% Pluronic F68 (PF68). The exact composition of the saline component of the buffer ranges from 160 mM to 180 mM NaCl. Optionally, a different pH buffer (potentially HEPES, sodium bicarbonate, TRIS) is used in place of the buffer specifically described. Still alternatively, a buffer containing 0.9% NaCl is useful.

Optionally, the compositions of the invention may contain, in addition to the rAAV and/or variants and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The pharmaceutical compositions containing at least one replication-defective rAAV virus, as described herein, can be formulated with a physiologically acceptable carrier, diluent, excipient and/or adjuvant, for use in gene transfer and gene therapy applications. In the case of AAV viral vectors, quantification of the genome copies (“GC”), vector genomes (“VG”), or virus particles may be used as the measure of the dose contained in the formulation or suspension. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome (usually poly A signal). In another method the effective dose of a recombinant adeno-associated virus carrying a nucleic acid sequence encoding the anti-PCSK9 antibody is measured as described in S. K. McLaughlin et al, 1988 J. Virol., 62:1963, which is incorporated by reference in its entirety.

As used herein, the term “dosage” can refer to the total dosage delivered to the subject in the course of treatment, or the amount delivered in a single unit (or multiple unit or split dosage) administration. All dosages may be measured by any known method, including by oqPCR or digital droplet PCR (ddPCR) as described in, e.g., M. Lock et al, Hum Gene Ther Methods. 2014 April; 25(2):115-25. doi: 10.1089/hgtb.2013.131, which is incorporated herein by reference. The replication-defective virus compositions can be formulated in dosage units to contain an amount of replication-defective virus that is in the range of about 1.0×10⁹ GC to about 1.0×10¹⁵ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰ , or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

These above doses may be administered in a variety of volumes of carrier, excipient or buffer formulation, ranging from about 25 microliters to about 1L, including all numbers within the range, depending on the size of the area to be treated, the viral titer used, the route of administration, and the desired effect of the method. In one embodiment, the volume of carrier, excipient or buffer is at least about 100 μL. In one embodiment, the volume is about 1 mL. In another embodiment, the volume is about 10 mL. In another embodiment, the volume is about 50 mL. In another embodiment, the volume is about 75 mL. In another embodiment, the volume is about 90 mL. In another embodiment, the volume is about 100 mL. In another embodiment, the volume is about 125 mL. In another embodiment, the volume is about 150 mL.

In one embodiment, the dosage is from about 1×10¹² GC/kg 1×10¹⁵ GC/kg of subject bodyweight, inclusive of endpoints. In one embodiment, the dosage is about 1×10¹⁴ GC/kg of subject bodyweight. In one embodiment, the dosage is about 1×10¹³ GC/kg of subject bodyweight. In one embodiment, the dosage is about 3×10¹³ GC/kg of subject bodyweight. In one embodiment, the dosage is about 3×10¹⁴ GC/kg of subject bodyweight.

In one embodiment, the viral constructs may be delivered in doses of from at least 1×10⁹to about least 1×10 ¹¹GCs in volumes of about 1 μL to about 25 μL for small animal subjects, such as mice. For larger veterinary subjects, the larger human dosages and volumes stated above are useful. In one embodiment, the viral constructs may be delivered in doses of from at least 1×10⁹ to about least 1×10¹⁶ GCs in volumes of about 50 mL to about 1000 mL. See, e.g., Diehl et al, J. Applied Toxicology, 21:15-23 (2001) for a discussion of good practices for administration of substances to various veterinary animals. This document is incorporated herein by reference.

The rAAV, preferably suspended in a physiologically compatible carrier, may be administered to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, maltose, and water. The selection of the carrier is not a limitation of the present invention. Optionally, the compositions of the invention may contain, in addition to the rAAV and carrier(s), excipient, diluent and/or adjuvant, other conventional pharmaceutical ingredients, such as preservatives, or chemical stabilizers.

The dosages, administrations and regimens may be determined by the attending physician given the teachings of this specification. In one embodiment, the composition is administered in a single dosage. In another embodiment, the composition is administered as a split dosage. Split administration may imply a time gap of administration from intervals of minutes, hours, days, weeks or months. In another embodiment, a second administration of an rAAV including the selected expression cassette (e.g., anti-PCSK9 antibody encoding cassette) is performed at a later time point. Such time point may be weeks, months or years following the first administration. In one embodiment, the second administration is 1, 2, 3, 4, 5, 6, 7, 8, 9, 10 years or more after the first administration. Such second administration is, in one embodiment, performed with an rAAV having a different capsid than the rAAV from the first administration. In another embodiment, the rAAV from the first and second administration have the same capsid.

In still other embodiments, the compositions described herein may be delivered in a single composition or multiple compositions. Optionally, two or more different AAV may be delivered, or multiple viruses [see, e.g., WO 2011/126808 and WO 2013/049493]. In another embodiment, multiple viruses may contain different replication-defective viruses (e.g., AAV and adenovirus or lentivirus).

In one embodiment, the compositions described herein are used in a method for lowering cholesterol in a subject. In still another embodiment, the compositions described herein are useful for increasing LDLr in a subject.

In a combination therapy, the AAV-delivered immunoglobulin construct described herein is administered before, during, or after commencing therapy with another agent, as well as any combination thereof, i.e., before and during, before and after, during and after, or before, during and after commencing cholesterol lowering therapy. Further, combinations of different AAV-delivered immunoglobulin constructs such as are discussed above may be used in such regimens. Thus, in one embodiment, the composition is administered before clinical symptoms of CVD or atherosclerosis, other than high LDL-C levels. Such elevated LDL-C levels may include levels greater than 130 mg/dl, greater than 140 mg/cl, greater than 150 mg/dl, greater than 160 mg/cl, greater than 170 mg/dl, greater than 180 mg/dl, greater than 190 mg/dl or higher. In another embodiment, the composition is administered after clinical symptoms of CVD or atherosclerosis.

Following administration of a dosage of a composition described in this specification, the subject is tested for efficacy of treatment, e.g., via measurement of LDL-C levels. See, e.g., Moriarty et al, Efficacy and safety of alirocumab, a monoclonal antibody to PCSK9, in statin-intolerant patients: Design and rationale of ODYSSEY ALTERNATIVE, a randomized phase 3 trial, J. Clinical Lapidology, 8(6):554-61 (November 2014) for a discussion of assessment of efficacy of anti-PSCK9 therapy. Such document is incorporated herein by reference.

In one embodiment, a method of generating a recombinant rAAV comprises obtaining a plasmid containing an AAV expression cassette as described above and culturing a packaging cell carrying the plasmid in the presence of sufficient viral sequences to permit packaging of the AAV viral genome into an infectious AAV envelope or capsid. Specific methods of rAAV vector generation are described above and may be employed in generating a rAAV vector that can deliver the novel anti-PSCK9 antibody sequences in the expression cassettes and genomes described above and in the examples below.

In yet another embodiment, a vector comprising any of the expression cassettes described herein is provided. As described above, such vectors can be plasmids of variety of origins and are useful in certain embodiments for the generation of recombinant replication defective viruses as described further herein.

In one another embodiment, the vector is a plasmid that comprises an expression cassette, wherein the expression cassette comprises AAV inverted terminal repeat sequences and a codon optimized nucleic acid sequence that encodes a novel anti-PSCK9 antibody, and expression control sequences that direct expression of the encoded immunoglobulin in a host cell.

As described above, the term “about” when used to modify a numerical value means a variation of ±10%, unless otherwise specified. As used throughout this specification and the claims, the terms “comprise” and “contain” and its variants including, “comprises”, “comprising”, “contains” and “containing”, among other variants, is inclusive of other components, elements, integers, steps and the like. The term “consists of” or “consisting of” are exclusive of other components, elements, integers, steps and the like.

The following examples are illustrative only and are not a limitation on the invention described herein. It is demonstrated herein, that a human antibody was murinized and administered to a mouse to effectively lower cholesterol levels in a model of familial hypercholesterolemia.

### EXAMPLE 1

### Construction of Human PCSK9mAb

A humanized mouse monoclonal antibody that targets PCSK9 has been previously been described. A human PCSK9mAb based on the available CDR regions of this published sequence was constructed that was then fused with constant heavy and light chain sequences. The heavy and light chain sequences were separated by a F2A linker that allowed for simultaneous expression of the heavy and light chain sequences encoded by a single polypeptide.

### EXAMPLE 2

### AAV8.TBG.hPCSK9mAb Administration Leads to Sustained Decrease in Serum Cholesterol

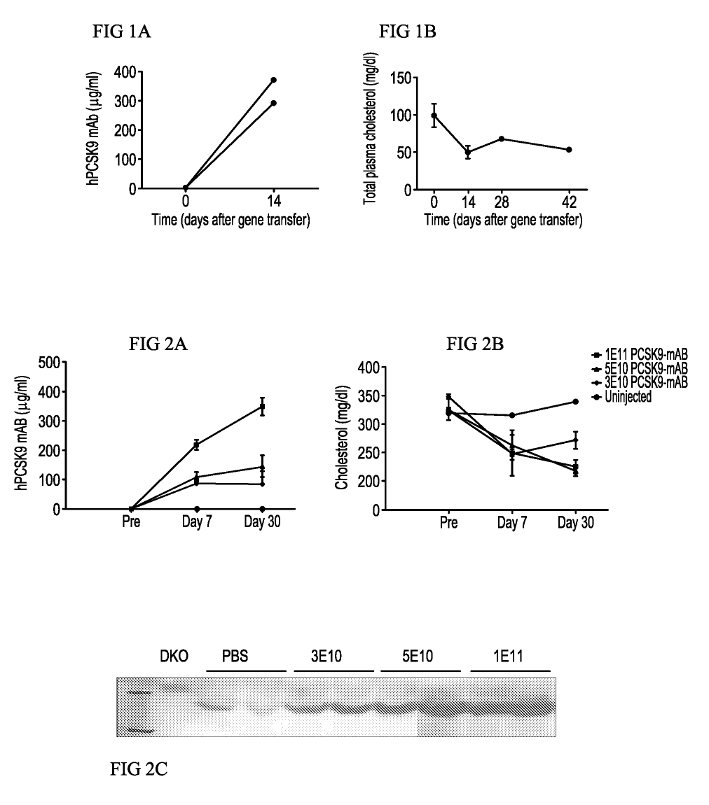

AV8.TBG.hPCSK9mAb was evaluated in C57B/6 mice (N=2) that express normal levels of endogenous mouse LDLR. Following intravenous administration hPCSK9mAB levels increased from undetectable levels to 300 ug/mL by day 14 (FIG. 1, A). Concomittantly, total plasma cholesterol levels decreased from their baseline levels of 100 mg/dl to 60 mg/dl. Cholesterol reductions were sustained and remained lower until day 42 (FIG. 1, B). These results demonstrate that AAV expressed PCSK9mAb causes stable decrease in plasma cholesterol levels over extended periods. These data also demonstrate the feasibility of using AAV expressed hPCSK9mAb in reducing total cholesterol in normal mice.

### EXAMPLE 3

### Evaluation of AAV9.hPCSK9mAb in LDLR+/−Mice

The effect of the hPCSK9mAb was evaluated in a humanized mouse model of familial hypercholesterolemia; a disease characterized by hypercholesterolemia. A mouse model lacking LDLR and APOBEC but transgenic for human Apolipoprotein B (LAHB) has been demonstrated to better recapitulate the human disease. See, Kassim et al, Adeno-Associated Virus Serotype 8 Gene Therapy Leads to Significant Lowering of Plasma Cholesterol Levels in Humanized Mouse Models of Homozygous and Heterozygous Familial Hypercholesterolemia, Human Gene Therapy, 24:19-26 (January 2013). As part of this study LAHB-LDLR haploinsufficient mice (LAHB LDLR +/−) were chosen to evaluate the dose response of AAVhPCSK9mAb. The presence of a single LDLR gene complement modestly increased the baseline serum cholesterol levels in these mice. This allowed better characterization of changes in LDLR expression that impact serum cholesterol levels following PCSK9 intervention.

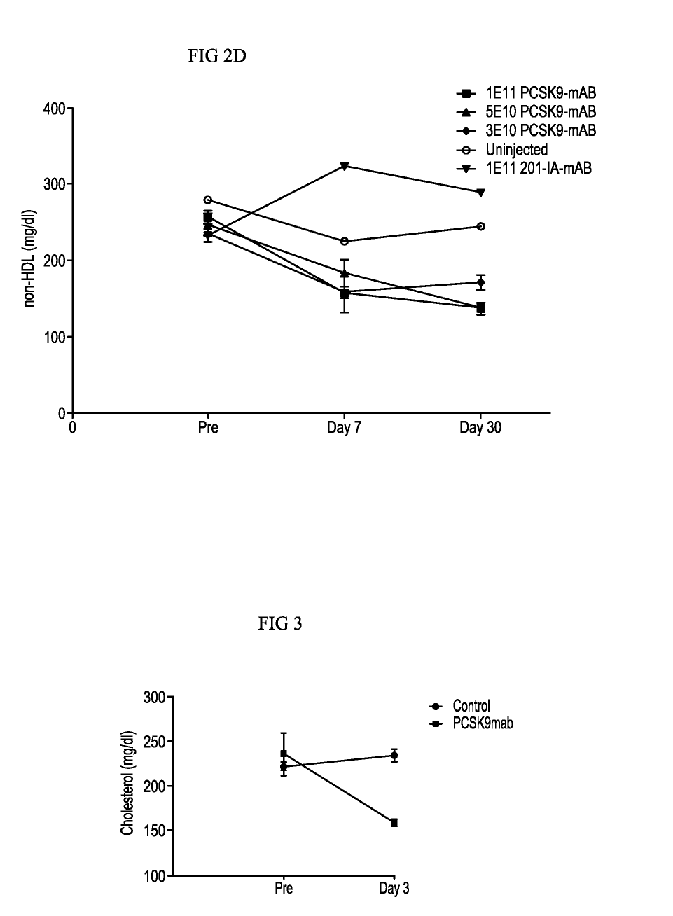

LAHB-LDLR-het mice were administered with increasing doses of AAVhPCSK9mAb. Vector administered mice demonstrated a dose dependent increase in hPCSK9 that peaked at day 30 (FIG. 2, A). Levels were about 350 ug/mL in the animals that received the highest dose (1×10¹¹ GC) of vector. More importantly, a 30% reduction in serum cholesterol was realized at this dose when compared to uninjected animals (FIG. 2, B). Immunoblotting of day 30 livers further confirmed that the decrease in cholesterol was accompanied by a dose dependent increase in the expression of endogenous mouse LDLR (FIG. 2, C).

### EXAMPLE 4

### Purified AAV Expressed hPCSKmAb is Functional and Lowers Cholesterol When Transferred to Mice

To further characterize the role of mAb, vector expressed antibody was purified from RAG−/− mice previously administered with AAVhPCSK9mAb and passively transferred to LAHB+/− mice. Reductions in serum cholesterol were noted three days after purified antibody administration when compared to uninjected mice (FIG. 3). These studies establish that AAV expressed hPCS9mAb causes sustained decrease in total cholesterol by upregulating LDLR expression in hepatocytes.

These studies demonstrate that AAV expressed human PCSK9mAb is functional and leads to sustained decrease in serum cholesterol levels by upregulating hepatic expression of the LDL receptor.

### EXAMPLE 5

### Coinjection of AAV-PCSK9 and AAV-LDLr

Mice were injected with 5×10¹⁰ of either AAV8 hLDLr, or AAV8 hLDLr -H306Y (Binds PCSK9 better), or AAV8 hLDLr -L318D (Hypothetically doesn't or poorly binds

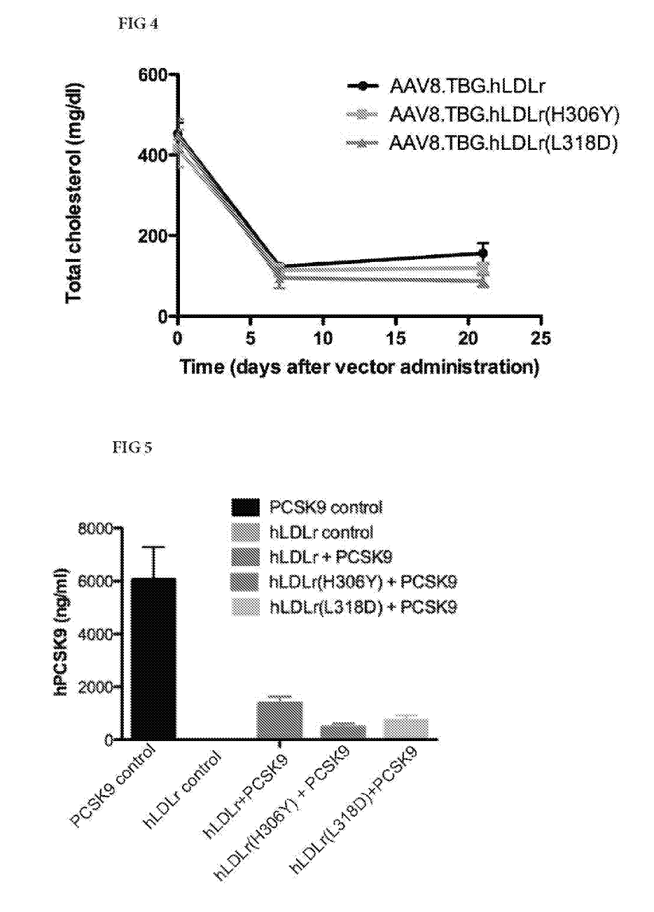

PCSK9). Then on day 42 mice would receive AAV9 PCSK9 at either a high dose of 1×10¹¹ or low of 1×10¹⁰ . Cholesterol was measured on days Baseline, Day 7, and Day 21. Both mutants reduced cholesterol equivalent to wt hLDLr. FIG. 4.

PCSK9 was assayed by ELISA and not detected. Serum was then assayed for neutralizing antibodies and the mice had at titer of at least 10. In summary both mutants reduce cholesterol levels. Re-administration should be done either before neutralizing antibody develops or potential at a later time point when levels may be reduced.

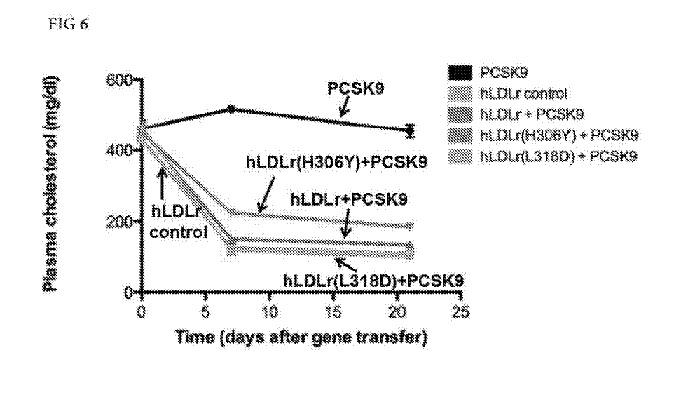

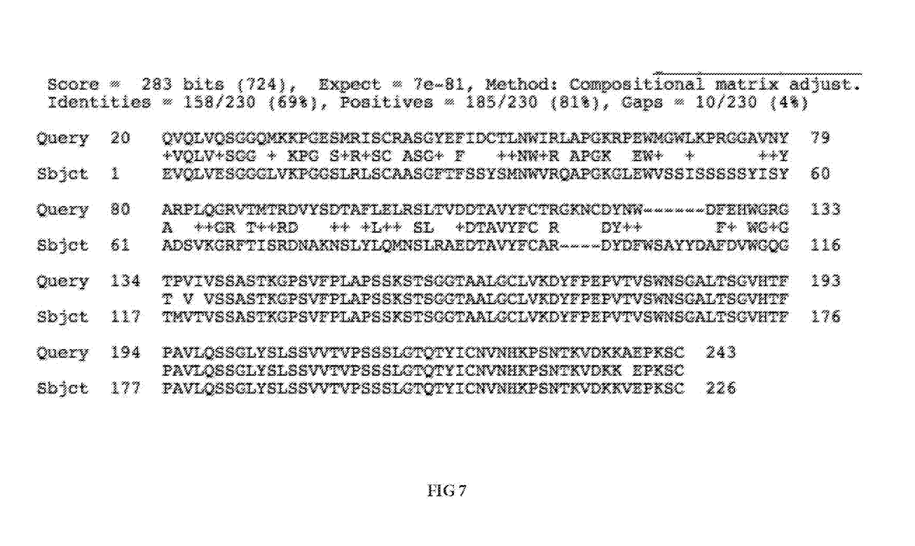

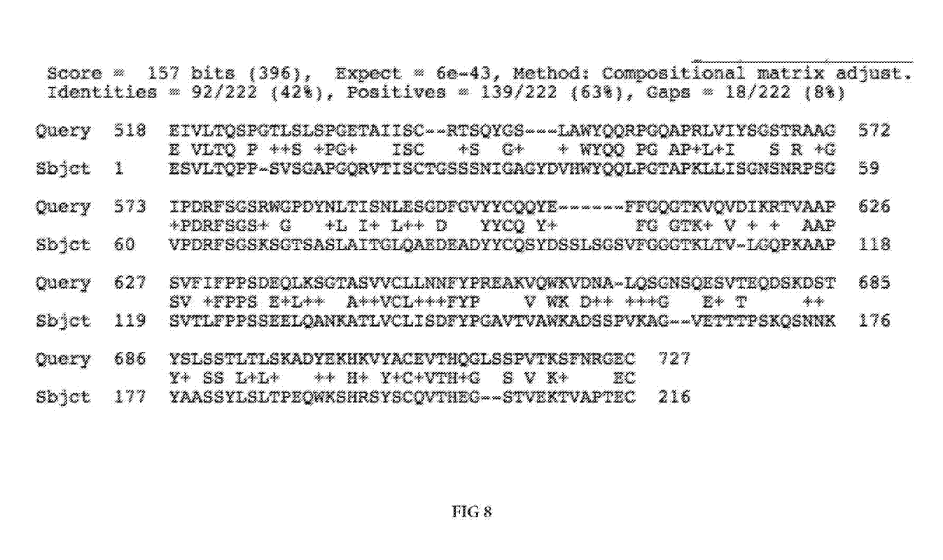

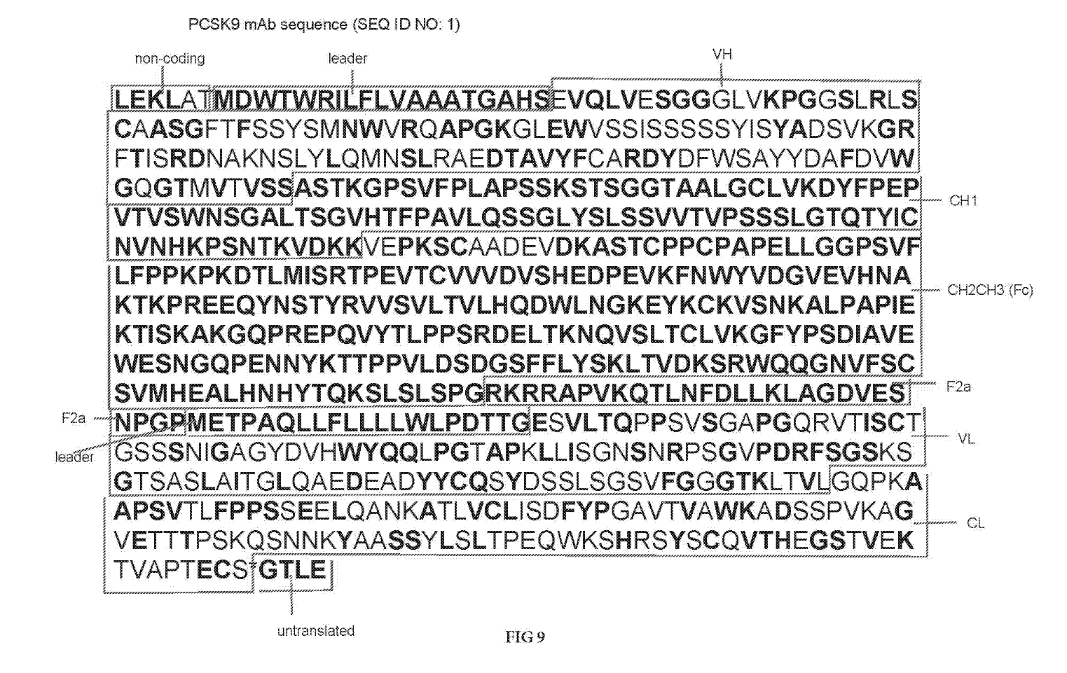

Mice received a co-injection of 3×10¹⁰ AAV9 PCSK9 and 5×10¹⁰ of either AAV8 hLDLr, or AAV8 hLDLr -H306Y (gain of function (GOF), Binds PCSK9 better), or AAV8 hLDLr-L318D (loss of function (LOF), Hypothetically doesn't or poorly binds PCSK9). A positive control for AAV9 PCSK9 only and AAV8 hLDLr only were also injected. PCSK9 expression was assayed with ELISA. The co-injection cohorts had a 3-8 fold decrease in expression compared to the control. FIG. 5. Cholesterol was measured at day 7 and 21. The GOF mutant had higher cholesterol levels than the other two cohorts, though not statistically significantly. All experienced a decrease in cholesterol levels. FIG. 6. In summary co-injection resulted in a lower level of PCSK9 expression than the positive control.

### EXAMPLE 6

### Methods

Animal studies: All animal studies were approved by the institutional review board (IRB) at the University of Pennsylvania. LDLR−/−, APOBEC-1−/−, human ApoB100 transgenic (LAHB) were maintained at the University of Pennsylvania. C57B/6 mice were purchased from Jackson Laboratories. All animals were fed a chow diet throughout the study. 6-8 week old male mice were injected intravenously (tail vein) with vector diluted in PBS in a total volume of 100 uL. Serum was collected pre and post vector administration by retro orbital bleeds. At the end of the study all animals were sacrificed and the livers harvested for analysis of vector genomes and transgene expression. Serum was analyzed for LDL, HDL and triglycerides using a MIRA analyzer (Roche).

Vector: The AAV vectors expressing hPCSK9mAb from a liver-specific thyroxine binding globulin (TBG) promoter (TBG) were obtained from the Vector Core at the University of Pennsylvania. Briefly, HEK293 cells were triple transfected using AAV cis-and trans-plasmid along with the Ad helper plasmids. AAV particles were purified from the culture supernatant and analyzed for DNA structure by restriction digests and endotoxin contamination (<20 EU/mL) before injection into animals.

Immunoblotting and Enzyme linked Immune assays: 75 ug of total cell lysates prepared from mouse livers were electrophoresed on a 4-12% gradient precast mini gel (Invitrogen) before transferring to PVDF membrane (Invitrogen). An anti-LDLR goat polyclonal antibody (Invitrogen) was used to probe the membrane ( 1/1000 dilution) followed by a secondary anti-goat antibody conjugated to alkaline phosphatase (Invitrogen). hPCSK9mAb expression levels in mouse serum were analyzed using an in-house ELISA. Briefly, ELISA plates were coated overnight with 2 ug/ml hPCSK9. Plates were washed, blocked and incubated with mouse serum for 1 hr at 37° C. Plates were developed using a biotin anti-human IgG secondary antibody followed by Strepavidin-HRP.

### EXAMPLE 7

### Administration of AAV9.TBG.hPCSK9.mAB to LDLR −/− Mice

LDLR −/−, APOBEC−/− double knockout (DKO) mice (N=5/group) were administered intravenously (tail vein) with 1E11 GC of an AAV9.TBG.hPCSK9.mAB vector. Control animals were injected at a similar dose with an AAV vector expressing a non-specific antibody against influenza (AAV9.TBG.FI6). Animals were bled retro-orbitally before and 30 days after vector administration. Serum cholesterol levels were evaluated using a MIRA analyzer (Roche). Non-HDL levels were calculated by subtracting the HDL levels from total cholesterol.

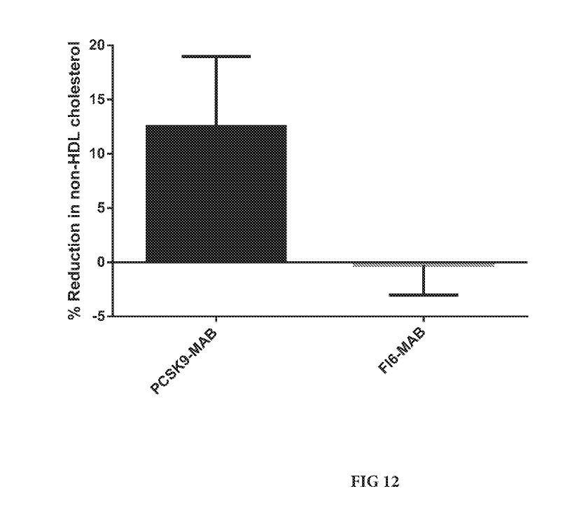

Previous studies (Example 3) with AAV9.PCSK9.mAB were indicative of cholesterol reductions when administered to mice haplo-insufficient for LDLR (LDLR+/−). As part of these studies we also dosed animals completely devoid of endogenous LDLR expression (LDLR−/−). In this model endogenous LDLR expression was knocked out by a process of homologous recombination and as such the animals do not express any detectable LDLR expression. However, administration of an AAV9 vector expressing an anti-PCSK9 monoclonal antibody from liver led to a significant (P<0.05) reduction in serum non-HDL cholesterol over baseline (12.51±3.2%, FIG. 12). Administration of a control vector expressing a non-specific antibody did not affect non-HDL cholesterol levels which remained at baseline (−0.29±1.2%).

The reduction in serum cholesterol observed following PCSK9.mAB administration was unexpected since these animals lack endogenous LDLR expression. A previous clinical trial was also ineffective in LDLR receptor negative familial hypercholesterolemia subjects dosed with a PCSK9.mAB (Circulation. 2013 November 5; 128(19):2113-20. Effect of the proprotein convertase subtilisin/kexin 9 monoclonal antibody, AMG 145, in homozygous familial hypercholesterolemia. Stein EA1, Honarpour N, Wasserman S M, Xu F, Scott R, Raal F J, which is incorporated herein by reference). However, other findings have demonstrated PCSK9 interaction with apolipoprotein B that prevents ApoB degradation and thereby leads to over production of triglyceride containing lipids (Arteriosclerosis, Thrombosis, and Vascular Biology. 2012; 32:1585-1595. Proprotein Convertase Subtilisin/Kexin Type 9 Interacts With Apolipoprotein B and Prevents Its Intracellular Degradation, Irrespective of the Low-Density Lipoprotein Receptor. Hua Sun, Amin Samarghandi, Ningyan Zhang, Zemin Yao, Momiao Xiong and Ba-Bie Teng and Circulation. 2014 Jul. 29; 130(5):431-41. Proprotein convertase subtilisin kexin type 9 promotes intestinal overproduction of triglyceride-rich apolipoprotein B lipoproteins through both low-density lipoprotein receptor-dependent and -independent mechanisms. Rashid S, Tavori H, Brown P E, Linton M F, He J, Giunzioni I, Fazio S, both of which are incorporated herein by reference). PCSK9 knockdown then would be expected to decrease serum cholesterol as was observed in our present study. It is possible that in the human trials the lack of steady state PCSK9.mAB levels may have led to a decreased effect on cholesterol levels. These findings suggest a novel approach for administering AAV.PCS9.mAB to both homozygous and heterozygous FH subjects to decrease serum cholesterol.

All publications cited in this specification, including provisional patent application No. 62/267,233, filed Dec. 14, 2015, are incorporated herein by reference in their entirety. Similarly, the SEQ ID Nos which are referenced herein and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

### Sequence Listing Free Text

| SEQ ID | |
|---|---|
| NO | FREE TEXT |
| 1 | <213> Artificial Sequence |
| | <223> constructed sequence |
| | <221> MISC_FEATURE |
| | <222> (1) . . . (6) |
| | <223> non-coding |
| | <220> |
| | <221> MISC_FEATURE |
| | <222> (7) . . . (25) |
| | <223> leader |
| | <220> |
| | <221> MISC_FEATURE |
| | <222> (26) . . . (148) |
| | <223> VH |
| | <220> |
| | <221> MISC_FEATURE |
| | <222> (149) . . . (245) |
| | <223> CH1 |
| | <220> |
| | <221> MISC_FEATURE |
| | <222> (246) . . . (482) |
| | <223> CH2—CH3 (Fc) |
| | <220> |
| | <221> MISC_FEATURE |
| | <222> (483) . . . (510) |
| | <223> F2a |
| | <220> |
| | <221> MISC_FEATURE |
| | <222> (511) . . . (530) |
| | <223> leader |
| | <220> |
| | <221> MISC_FEATURE |
| | <222> (531) . . . (641) |
| | <223> VL |
| | <220> |
| | <221> MISC_FEATURE |
| | <222> (642) . . . (747) |
| | <223> CL |
| | <220> |
| | <221> MISC_FEATURE |
| | <222> (748) . . . (751) |
| | <223> untranslated |
| | <213> Artificial Sequence |
| | <220> |
| 2 | <223> constructed sequence |
| | <213> Artificial Sequence |
| | <220> |
| 3 | <223> constructed sequence |
| | <213> Artificial Sequence |
| | <220> |
| 4 | <223> constructed sequence |
| | <213> Artificial Sequence |
| | <220> |
| 5 | <223> constructed sequence |
| | <213> Artificial Sequence |
| | <220> |
| 6 | <223> constructed sequence |
| | <213> Artificial Sequence |
| | <220> |
| 7 | <223> constructed sequence |
| | <213> Artificial Sequence |
| | <220> |
| 8 | <223> constructed sequence |
| | <213> Artificial Sequence |
| | <220> |
| 9 | <223> constructed sequence |
| | <213> Artificial Sequence |
| | <220> |
| 10 | <223> constructed sequence |

1. An adeno-associated viral (AAV) vector comprising an AAV capsid and at least one expression cassette, wherein the expression cassettes comprise nucleic acid sequences encoding an anti-PCSK9 antibody and expression control sequences that direct expression of the anti-PCSK9 antibody sequences in a host cell.

2. The AAV vector of claim 1, wherein the antibody has the sequence of SEQ ID NO: 1, or a sequence sharing at least 95% identity therewith.

3. The AAV vector of claim 1, wherein the antibody is encoded by SEQ ID NO: 8, or a sequence sharing at least 70% identity therewith.

4. The AAV vector of claim 1, wherein the antibody comprises one or more of
a constant heavy chain sequence having the sequence of SEQ ID NO: 2,
a constant light chain sequence having the sequence of SEQ ID NO: 3,
a variable heavy chain sequence having the sequence of SEQ ID NO: 4, and
a variable light chain sequence having the sequence of SEQ ID NO: 5.

5-9. (canceled)

10. The AAV vector of claim 1, wherein the anti-PCSK9 antibody is selected from evolocumab, bococizumab, alirocumab, and LGT209.

11-15. (canceled)

16. The viral vector according to claim 1, wherein said nucleic acid sequences encoding the anti-PCSK9 chimeric antibody comprise SEQ ID NO: 6 or a sequence sharing at least 70% identity therewith.

17. The viral vector according to claim 16, wherein the sequence sharing at least 70% identity with SEQ ID NO: 6 is a codon optimized sequence.

18. A viral vector of claim 1, wherein said expression cassettes comprise nucleic acid sequences encoding one or more the anti-PCSK9 heavy chain sequence of SEQ ID NO: 11 and the anti-PCSK9 light chain sequence of SEQ ID NO: 12 and expression control sequences that direct expression of an anti-PSCK9 antibody comprising the variable and light chain sequences in a host cell.

19. A viral vector of claim 1, wherein said expression cassettes comprise nucleic acid sequences encoding one or more the anti-PCSK9 heavy chain sequence of SEQ ID NO: 13 and the anti-PCSK9 light chain sequence of SEQ ID NO: 14 and expression control sequences that direct expression of an anti-PSCK9 antibody comprising the variable and light chain sequences in a host cell.

20. The viral vector of claim 1, wherein the expression control sequences comprise a promoter selected from a CMV promoter, a TBG promoter, a tissue-specific promoter, a muscle-specific promoter, or a liver-specific promoter.

21-27. (canceled)

28. The viral vector of claim 1, wherein the vector is a rAAV having a capsid selected from AAV8, rh64R1, AAV9, AAVhu.37, or rh10 and variants thereof.

29. (canceled)

30. The viral vector of claim 28, wherein the ITRs are from an AAV different from the AAV supplying the capsid.

31. The viral vector of claim 30, wherein the ITRs are from AAV2.

32. (canceled)

33. A pharmaceutical composition comprising a pharmaceutically acceptable carrier and at least a viral vector according to claim 1.

34. A chimeric antibody comprising the sequence of SEQ ID NO: 1, or a sequence sharing 95% identity therewith, a constant heavy chain sequence of SEQ ID NO: 4, or a constant light chain sequence of SEQ ID NO: 5.

35-37. (canceled)

38. A pharmaceutical composition comprising the antibody according to claim 34 and a pharmaceutically acceptable carrier, excipient, diluent and/or adjuvant.

39. A method for lowering cholesterol or increasing LDLr expression in a subject, said method comprising administering the composition of claim 33 to a subject in need thereof.

40. A method for lowering cholesterol or increasing LDLr expression in a subject, said method comprising administering the composition of claim 38 to a subject in need thereof.

41-43. (canceled)

44. The method according to claim 39, wherein said composition is administered in combination with a statin, LDLR, or an AAV vector which comprises an expression cassette which encodes LDLR.

45. The method according to claim 40, wherein the composition is administered with a statin, LDLR, or an AAV vector which comprises an expression cassette which encodes LDLR.

46-69. (canceled)


SEQUENCE LISTING (14 sequences)

SEQ ID NO: 1 - protein, 751 aa, Artificial Sequence
  constructed sequence
  MISC_FEATURE (1)..(6): non-coding
  MISC_FEATURE (7)..(25): leader
  MISC_FEATURE (26)..(148): VH
  MISC_FEATURE (149)..(245): CH1
  MISC_FEATURE (246)..(482): CH2-CH3 (Fc)
  MISC_FEATURE (483)..(510): F2a
  MISC_FEATURE (511)..(530): leader
  MISC_FEATURE (531)..(641): VL
  MISC_FEATURE (642)..(747): CL
  MISC_FEATURE (748)..(751): untranslated
LEKLATMDWTWRILFLVAAATGAHSEVQLVESGGGLVKPGGSLRLSCAASGFTFSSYSMNWVRQAPGKGLEWVSSISSSSSYISYADSVKGRFTISRDNAKNSLYLQMNSLRAEDTAVYFCARDYDFWSAYYDAFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAADEVDKASTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGRKRRAPVKQTLNFDLLKLAGDVESNPGPMETPAQLLFLLLLWLPDTTGESVLTQPPSVSGAPGQRVTISCTGSSSNIGAGYDVHWYQQLPGTAPKLLISGNSNRPSGVPDRFSGSKSGTSASLAITGLQAEDEADYYCQSYDSSLSGSVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGTLE

SEQ ID NO: 2 - protein, 334 aa, Artificial Sequence
  constructed sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAADEVDKASTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 106 aa, Artificial Sequence
  constructed sequence
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 4 - protein, 123 aa, Artificial Sequence
  constructed sequence
EVQLVESGGGLVKPGGSLRLSCAASGFTFSSYSMNWVRQAPGKGLEWVSSISSSSSYISYADSVKGRFTISRDNAKNSLYLQMNSLRAEDTAVYFCARDYDFWSAYYDAFDVWGQGTMVTVSS

SEQ ID NO: 5 - protein, 111 aa, Artificial Sequence
  constructed sequence
ESVLTQPPSVSGAPGQRVTISCTGSSSNIGAGYDVHWYQQLPGTAPKLLISGNSNRPSGVPDRFSGSKSGTSASLAITGLQAEDEADYYCQSYDSSLSGSVFGGGTKLTVL

SEQ ID NO: 6 - protein, 19 aa, Artificial Sequence
  constructed sequence
MDWTWRILFLVAAATGAHS

SEQ ID NO: 7 - protein, 20 aa, Artificial Sequence
  constructed sequence
METPAQLLFLLLLWLPDTTG

SEQ ID NO: 8 - DNA, 2229 bp, Artificial Sequence
  constructed sequence
atggactggacctggcggatcctgttcctggtggctgctgcaacaggcgcccactctgaagtgcagctggtggaatctggcggcggactggtcaaacctggcggctctctgagactgagctgtgccgccagcggcttcaccttcagcagctacagcatgaactgggtccgacaggcccctggcaagggactggaatgggtgtccagcatcagcagcagcagctcctacatcagctacgccgacagcgtgaagggccggttcaccatcagccgggacaacgccaagaacagcctgtacctgcagatgaacagcctgcgggccgaggataccgccgtgtacttctgcgccagagactacgacttttggagcgcctactacgacgccttcgacgtgtggggccagggcacaatggtcacagtgtctagcgccagcaccaagggcccctccgtgtttcctctggcccctagcagcaagagcacctctggcggaacagccgccctgggatgcctggtcaaggactacttccccgagcccgtgaccgtgtcctggaactctggcgccctgacaagcggcgtgcacacatttccagccgtgctgcagagcagcggcctgtactctctgagcagcgtggtcaccgtgccctcttctagcctgggcacccagacctacatctgcaacgtgaaccacaagcccagcaacaccaaggtggacaagaaggtggaacccaagtcctgtgccgccgacgaggtggacaaagccagcacctgtcccccttgtcctgcccctgaactgctgggaggccctagcgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaatgccaagaccaagcccagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagcccagagaaccccaggtgtacacactgccccccagcagagatgagctgaccaagaaccaggtgtccctgacctgcctcgtgaagggcttctaccccagcgatatcgccgtggaatgggagagcaacggccagcctgagaacaactacaagaccaccccccctgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagccccggcagaaaaagacgggcccctgtgaagcagaccctgaacttcgacctgctgaagctggccggcgacgtggaaagcaaccctggccctatggaaacccctgcccagctgctgtttctgctgctgctgtggctgcccgataccacaggcgagagcgtgctgacacagcccccatctgtgtctggcgcccctggacagagagtgaccatcagctgtaccggcagctccagcaacatcggagccggctacgacgtgcactggtatcagcagctgcctggcaccgcccccaagctgctgatcagcggcaacagcaacagacctagcggcgtgcccgacagattcagcggcagcaagtctggcacctctgccagcctggccatcactggactgcaggccgaggacgaggccgactactactgccagtcctacgacagcagcctgagcggctccgtgtttggcggcggaacaaaactgaccgtgctgggccagcctaaggccgctcctagcgtgaccctgtttccacccagcagcgaggaactgcaggccaacaaagccaccctcgtgtgcctgatcagcgacttttaccctggcgccgtgacagtggcttggaaggccgatagcagccctgtgaaggccggcgtggaaaccaccacccctagcaagcagtccaacaacaaatacgccgccagcagctacctgtctctgacccccgagcagtggaagtcccacagatcctacagctgccaggtcacacacgagggcagcaccgtggaaaagaccgtggcccctaccgagtgcagctgatga

SEQ ID NO: 9 - DNA, 6796 bp, Artificial Sequence
  constructed sequence
ggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaagcttgccaccatggactggacctggcggatcctgttcctggtggctgctgcaacaggcgcccactctgaagtgcagctggtggaatctggcggcggactggtcaaacctggcggctctctgagactgagctgtgccgccagcggcttcaccttcagcagctacagcatgaactgggtccgacaggcccctggcaagggactggaatgggtgtccagcatcagcagcagcagctcctacatcagctacgccgacagcgtgaagggccggttcaccatcagccgggacaacgccaagaacagcctgtacctgcagatgaacagcctgcgggccgaggataccgccgtgtacttctgcgccagagactacgacttttggagcgcctactacgacgccttcgacgtgtggggccagggcacaatggtcacagtgtctagcgccagcaccaagggcccctccgtgtttcctctggcccctagcagcaagagcacctctggcggaacagccgccctgggatgcctggtcaaggactacttccccgagcccgtgaccgtgtcctggaactctggcgccctgacaagcggcgtgcacacatttccagccgtgctgcagagcagcggcctgtactctctgagcagcgtggtcaccgtgccctcttctagcctgggcacccagacctacatctgcaacgtgaaccacaagcccagcaacaccaaggtggacaagaaggtggaacccaagtcctgtgccgccgacgaggtggacaaagccagcacctgtcccccttgtcctgcccctgaactgctgggaggccctagcgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaatgccaagaccaagcccagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagcccagagaaccccaggtgtacacactgccccccagcagagatgagctgaccaagaaccaggtgtccctgacctgcctcgtgaagggcttctaccccagcgatatcgccgtggaatgggagagcaacggccagcctgagaacaactacaagaccaccccccctgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagccccggcagaaaaagacgggcccctgtgaagcagaccctgaacttcgacctgctgaagctggccggcgacgtggaaagcaaccctggccctatggaaacccctgcccagctgctgtttctgctgctgctgtggctgcccgataccacaggcgagagcgtgctgacacagcccccatctgtgtctggcgcccctggacagagagtgaccatcagctgtaccggcagctccagcaacatcggagccggctacgacgtgcactggtatcagcagctgcctggcaccgcccccaagctgctgatcagcggcaacagcaacagacctagcggcgtgcccgacagattcagcggcagcaagtctggcacctctgccagcctggccatcactggactgcaggccgaggacgaggccgactactactgccagtcctacgacagcagcctgagcggctccgtgtttggcggcggaacaaaactgaccgtgctgggccagcctaaggccgctcctagcgtgaccctgtttccacccagcagcgaggaactgcaggccaacaaagccaccctcgtgtgcctgatcagcgacttttaccctggcgccgtgacagtggcttggaaggccgatagcagccctgtgaaggccggcgtggaaaccaccacccctagcaagcagtccaacaacaaatacgccgccagcagctacctgtctctgacccccgagcagtggaagtcccacagatcctacagctgccaggtcacacacgagggcagcaccgtggaaaagaccgtggcccctaccgagtgcagctgatgaggtaccctcgagaattcacgcgtggtacctctagagtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaa

SEQ ID NO: 10 - DNA, 6679 bp, Artificial Sequence
  constructed sequence
aattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaagcttgccaccatggactggacctggcggatcctgttcctggtggctgctgcaacaggcgcccactctgaagtgcagctggtggaatctggcggcggactggtcaaacctggcggctctctgagactgagctgtgccgccagcggcttcaccttcagcagctacagcatgaactgggtccgacaggcccctggcaagggactggaatgggtgtccagcatcagcagcagcagctcctacatcagctacgccgacagcgtgaagggccggttcaccatcagccgggacaacgccaagaacagcctgtacctgcagatgaacagcctgcgggccgaggataccgccgtgtacttctgcgccagagactacgacttttggagcgcctactacgacgccttcgacgtgtggggccagggcacaatggtcacagtgtctagcgccagcaccaagggcccctccgtgtttcctctggcccctagcagcaagagcacctctggcggaacagccgccctgggatgcctggtcaaggactacttccccgagcccgtgaccgtgtcctggaactctggcgccctgacaagcggcgtgcacacatttccagccgtgctgcagagcagcggcctgtactctctgagcagcgtggtcaccgtgccctcttctagcctgggcacccagacctacatctgcaacgtgaaccacaagcccagcaacaccaaggtggacaagaaggtggaacccaagtcctgtgccgccgacgaggtggacaaagccagcacctgtcccccttgtcctgcccctgaactgctgggaggccctagcgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaatgccaagaccaagcccagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagcccagagaaccccaggtgtacacactgccccccagcagagatgagctgaccaagaaccaggtgtccctgacctgcctcgtgaagggcttctaccccagcgatatcgccgtggaatgggagagcaacggccagcctgagaacaactacaagaccaccccccctgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagccccggcagaaaaagacgggcccctgtgaagcagaccctgaacttcgacctgctgaagctggccggcgacgtggaaagcaaccctggccctatggaaacccctgcccagctgctgtttctgctgctgctgtggctgcccgataccacaggcgagagcgtgctgacacagcccccatctgtgtctggcgcccctggacagagagtgaccatcagctgtaccggcagctccagcaacatcggagccggctacgacgtgcactggtatcagcagctgcctggcaccgcccccaagctgctgatcagcggcaacagcaacagacctagcggcgtgcccgacagattcagcggcagcaagtctggcacctctgccagcctggccatcactggactgcaggccgaggacgaggccgactactactgccagtcctacgacagcagcctgagcggctccgtgtttggcggcggaacaaaactgaccgtgctgggccagcctaaggccgctcctagcgtgaccctgtttccacccagcagcgaggaactgcaggccaacaaagccaccctcgtgtgcctgatcagcgacttttaccctggcgccgtgacagtggcttggaaggccgatagcagccctgtgaaggccggcgtggaaaccaccacccctagcaagcagtccaacaacaaatacgccgccagcagctacctgtctctgacccccgagcagtggaagtcccacagatcctacagctgccaggtcacacacgagggcagcaccgtggaaaagaccgtggcccctaccgagtgcagctgatgaggtaccctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctag

SEQ ID NO: 11 - protein, 447 aa, Homo sapiens
EVQLVESGGGLVQPGGSLRLSCAASGFTFNNYAMNWVRQAPGKGLDWVSTISGSGGTTNYADSVKGRFIISRDSSKHTLYLQMNSLRAEDTAVYYCAKDSNWGNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 220 aa, Homo sapiens
DIVMTQSPDSLAVSLGERATINCKSSQSVLYRSNNRNFLGWYQQKPGQPPNLLIYWASTRESGVPDRFSGSGSGTDFTLTISSLQAEDVAVYYCQQYYTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 441 aa, Homo sapiens
EVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 14 - protein, 215 aa, Homo sapiens
ESALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS